(19) United States
(12) Patent Application Publication
(10) Pub. No.: US 2025/0010050 A1
(43) Pub. Date: Jan. 9, 2025

(54) MICROPROJECTION ARRAYS WITH ENHANCED SKIN PENETRATING PROPERTIES AND METHODS THEREOF

(71) Applicant: Vaxxas Pty Limited, Sydney (AU)

(72) Inventors: Stefano MELIGA, West End (AU); Mark Anthony Fernance KENDALL, Chelmer (AU); Robert William GODDARD, Capalaba (AU)

(21) Appl. No.: 18/886,905

(22) Filed: Sep. 16, 2024

Related U.S. Application Data
(63) Continuation of application No. 17/241,927, filed on Apr. 27, 2021, now Pat. No. 12,090,295.
Application No. 17/241,927 is a continuation of application No. 15/762,913, filed on Mar. 23, 2018, now abandoned.
(60) Provisional application No. 62/233,607, filed on Sep. 28, 2015.

(51) Int. Cl. A61M 37/00; A61B 17/20; A61D 7/00

(52) U.S. Cl. CPC A61M 37/0015; A61B 17/205; A61D 7/00; A61M 2037/0023; A61M 2037/0046; A61M 2037/0061; A61M 2202/30; A61M 2207/00; A61M 2210/04

(57) ABSTRACT

An apparatus for delivering an active ingredient into the skin of an animal at a defined depth, the apparatus including: a microprojection array including a plurality of microprojections having a density of at least 2,000 projections per cm²; and an applicator that drives the microprojection array towards the skin in use so that the microprojection array impacts on the skin with a mass-to-velocity ratio of between 0.0005 g/m/s and 0.1 g/m/s per cm².

### BACKGROUND OF THE INVENTION

The invention is generally directed to devices and methods for intradermal delivery of active agents into the skin, more particularly the invention is directed to devices and methods for improving the immunogenicity of vaccine preparations by intradermal delivery of the vaccine via a microprojection array in which the parameters for delivery of the active agents have been developed to achieve appropriate depth penetration and efficient delivery of the active agent.

### DESCRIPTION OF THE PRIOR ART

Next-generation healthcare increasingly relies on minimally-invasive biomedical devices capable of negotiating skin mechanical properties to mediate intracutaneous and transcutaneous tasks like administering therapeutics, extracting diagnostic biomarkers and performing surgical procedures. For instance, epidermal and dermal targeted delivery of vaccines is a promising candidate for increasing global vaccine coverage, due to case of access as well as unique immunological properties of the skin. Passive permeation of the antigen is impractical due to the large molecular size of most antigens, therefore, the payload is actively transported to the viable-cell strata by mechanically breaching through the skin's outer barriers. This transport is typically achieved by either: 1) high-pressure jet injectors that fire the payload in liquid or powder form (microparticles) or 2) penetrator tips that deposit payload through a channel in the skin (e.g. intradermal syringe needles and hollow microneedles), or that embed the payload in a matrix/coating that dissolves in the skin (e.g. dissolvable/coated microneedle and microprojection arrays). Some studies have reported improved immune responses compared to standard syringe injection. In addition, the mechanisms underlying the low-dose efficacy or increased potency are not yet fully understood thereby limiting the potential of cutaneous vaccination.

Precise penetration to the targeted depth for vaccine uptake by site-specific cells is of fundamental importance and relies on negotiating the unique elastic and failure properties of the skin which is a multilayer composite ‘material’. Despite the many published mechanical characterization and underlying linear and non-linear elastic models, there is a paucity of investigations focusing on skin elastic and failure behavior in mechanical conditions relevant for epidermal and dermal delivery of active agents including vaccines. There are reasons beyond the skin's intrinsic structural complexity, and inter-species (e.g. mouse vs human), inter-individual (ethnicity, gender) and intra-individual (age, body site) variabilities for this failure. Firstly, the persistent assumption of skin homogeneity and isotropicity resulted in different clastic moduli depending on the loading mode. Secondly, the Young's moduli extrapolated from indentations showed a marked inverse dependence with the probe diameter. Thirdly, although the extensive literature on skin viscoelasticity provides solid evidence of the rate-dependence of skin elasticity, there appear to be no published out-of-plane tests where the load was applied at velocities >1 m s⁻¹ or strain rates >1 μs⁻¹.

While underlying linear-elastic and hyperelastic descriptions are corroborated by empirical data, skin also lacks established constitutive models of failure. Skin penetration by individual needles has typically been described using either: 1) stress-based failure criteria extend the traditional yield criteria such that the skin fails when the stress (typically the von Mises component) exceeds a threshold strength; as such, this framework does not account for the irrecoverable energy dissipated into material damage and, thus, for example, cannot be used to predict the depth achieved by penetrators fired at a given velocity; or 2) energy-based fracture propagation extends the concept of fracture toughness to ductile materials, i.e. an energy per unit area representing the cost to create crack interfaces. This model, though, does not specify if an initial notch forms at all (failure initiation), how the crack propagates (e.g. direction and speed), and what fraction of the penetrator energy is utilized in the fracture (as opposed of being elastically stored or dissipated in viscous or plastic phenomena). Rather, the prediction of skin penetration requires a complete description of the spatial stress-strain distributions to detect the instant and coordinates of failure initiation, and the energy repartition among various reversible and irreversible phenomena.

Skin out-of-plane mechanical properties of skin at the microscale are typically measured ex vivo using indentation (e.g. AFM) at velocities up to ˜100 μm s⁻¹; however, vaccines are delivered in vivo across the skin's superficial barriers using penetrators applied (by hand or impact applicators) at velocities >>mm s⁻¹; strain-rate effects and subcutaneous layers play an important mechanical role during skin penetration.

The limited understanding of skin elastic response to high strain rates, mechanisms of failure and fracture, and interaction with multiple penetrators have prevented the rational design of epidermal and dermal targeted vaccination devices. Some microprojection arrays are silicon chips containing, on one side, thousands of densely-arranged (>>1,000 cm⁻²) microprojections, i.e. solid cone-like structures measuring ˜100 μm in length. Notably, application of vaccine-coated microprojection arrays to mouse skin elicited immune response using ˜ 1/100 of the dose required by intramuscular injection. The precise and consistent targeting of specific strata within the skin is important and achieved by applying the array against the skin at controlled velocities (˜1 m s⁻¹). Therefore, there is a need for in-depth understanding of the skin mechanical interaction with microneedles/microprojections which would allow the tailoring of an array design and application conditions to achieve customized antigen placement and to increase the targeting consistency across patients and minimize the penetration energy of the array while controlling skin inflammation, tolerability and acceptability.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

### SUMMARY OF THE PRESENT INVENTION

In a broad form the present invention seeks to provide an apparatus for delivering an active ingredient into the skin of an animal at a defined depth, the apparatus including:
a) a microprojection array including a plurality of microprojections having a density of at least 2,000 projections per cm²; and,
b) an applicator that drives the microprojection array towards the skin in use so that the microprojection array impacts on the skin with a mass-to-velocity ratio of between 0.0005 g/m/s and 0.1 g/m/s per cm².

Typically the microprojection array impacts on the skin with a mass-to-velocity ratio of at least one of:
a) less than 0.05 g/m/s;
b) less than 0.005 g/m/s; and,
c) between 0.033 g/m/s and 0.0008 g/m/s.

Typically the microprojection array impacts the skin with a mass between at least one of:
a) 0.001 g and 5 g;
b) 0.005 g and 2 g; and,
c) 0.02 g and 0.5 g.

Typically the microprojection array impacts the skin at velocities between:
a) 5 m/s and 50 m/s;
b) 10 m/s g and 30 m/s; and,
c) 15 m/s and 25 m/s.

Typically the microprojection array has an area between at least one of:
a) 16 mm² and 400 mm²;
b) 36 mm² and 225 mm²; and,
c) 64 mm² and 100 mm².

Typically the microprojection array has a microprojection density between 5,000 and 20,000 projections per cm².

Typically the microprojections are at least one of:
a) solid;
b) non-porous; and,
c) non-hollow.

Typically the microprojections are at least one of:
a) tapered;
b) substantially conical;
c) substantially flattened;
d) hexagonal; and,
e) octagonal.

Typically the microprojections have a length of at least one of:
a) more than 100 μm;
b) more than 200 μm;
c) less than 1000 μm;
d) less than 5000 μm; and,
e) between 200 μm and 300 μm.

Typically the microprojections include:
a) a base having a width of about 5 μm to about 50 μm; and,
b) a tip having a width of about 0.5 μm to about 2 μm.

Typically the applicator includes a driver that drives the microprojection array towards the skin and wherein the microprojection array is releasably mounted to the driver so that the microprojection array is released from the driver prior to the microprojections contacting the skin.

Typically the driver abuts against a stop to thereby release the microprojection array.

Typically the stop includes an annular shoulder.

Typically the applicator includes:
a) a housing containing the driver; and,
b) a substantially tubular spacer that in use is positioned with an open end in contact with a surface of the skin to thereby space the housing from the skin, the stop being provided proximate the open end of the spacer.

Typically the driver is urged from a retracted to an extended position using a biasing mechanism, and wherein the biasing mechanism and engagement between the driver and housing define a driver velocity in use.

Typically the driver is a piston.

Typically the biasing mechanism includes at least one of:
a) a spring; and,
b) a pneumatic actuator.

Typically the engagement is frictional engagement between a piston and piston chamber within the housing.

Typically the microprojection array impacts on the skin with a mass-to-velocity ratio sufficiently high to effect at least one of:
a) fracture the skin;
b) concentrate mechanical stress in superficial layers of the skin;
c) invoke strain-rate dependent skin stiffening;
d) cause consistent penetration independent of variations in subcutaneous properties of the skin;
e) dissipate inertia so as to avoid mechanical stress on body parts underlying the skin; and,
f) cause a controlled amount of mechanical stress for immune-enhancing inflammation.

Typically at least tips of the microprojections are coated.

Typically the active ingredient is one or more vaccine antigens.

In another broad form the present invention seeks to provide a method of determining the design of a microprojection array and the velocity for delivering the microprojection array to a predetermined range of skin depth comprising calculating the microprojection array density, microprojection array area, microprojection array mass and microprojection velocity to mass ratio to deliver the microprojection array to the predetermined depth range.

### BRIEF DESCRIPTION OF THE DRAWINGS

An example of the present invention will now be described with reference to the accompanying drawings, in which:

FIG. 1A is a schematic drawing of various modes of penetrating the skin;

FIG. 1B is a schematic diagram of design specifications for individual and arrays of penetrators (e.g. microneedles/microprojections);

FIG. 1C is a schematic drawing of a mouse ear section and skin layer thickness;

FIGS. 2A-2H are graphical representations of the hyperelastic properties for the skin layers (SC=stratum corneum, VE=viable epidermis, dermis) of mouse car as a function of indentation velocity (or peak logarithmic strain rate): FIG. 2A is a plot of Young's moduli versus the strain rate and velocity for the stratum corneum; FIG. 2B is a plot of Young's moduli versus the strain rate and velocity for the viable epidermis; FIG. 2C is a plot of Young's moduli versus the strain rate and velocity for the dermis; FIG. 2D is a plot of the stretch exponent versus the strain rate and velocity for the stratum corneum; FIG. 2E is a plot of the stretch exponent versus the strain rate and velocity for the viable epidermis; FIG. 2F is a plot of the stretch exponent versus the strain rate and velocity for the dermis; and FIGS. 2G and 2H are bar diagrams of Young's modulus and stretch exponent extrapolated for a probe measuring 1 μm in diameter indenting the skin layers in the velocity range 0.3-10 m s⁻¹ (or strain-rate range 0.3-10 μs⁻¹);

FIGS. 3A-3D are graphical representations of skin stress and energy transfers during the penetration by arrays with different densities applied with equal energy per projection (˜½*35 g*(2 m s⁻¹)²/3000): FIG. 3A shows VM stress in the skin during the penetration of arrays characterized by projection densities of ˜0 proj cm⁻² (infinitely-spaced projections); FIG. 3B shows VM stress in the skin during the penetration of arrays characterized by projection densities of 5,000 proj cm⁻²; FIG. 3C shows VM stress in the skin during the penetration of arrays characterized by projection densities of 10,000 proj cm⁻²; and FIG. 3D shows VM stress in the skin during the penetration of arrays characterized by projection densities of 20,400 proj cm⁻²;

FIG. 3E is a diagram of symmetric FE geometry and mesh used to simulate the penetration of arrays with ≥5,000 proj cm⁻², in which the inset shows the fundamental skin unit simulated (red) and the planes of symmetry (dashed lines) on a top-view schematics of the array;

FIG. 3F is a diagram of the fraction of application energy (mean±range) utilized during the penetration of the ˜0 proj cm⁻² array into mouse car when the tip reaches the bottom of the (ventral) dermis as calculated using FEM; the range represent the variation between successive time points (±0.5 μs);

FIG. 3G is a diagram of the energy utilized as function of projection density/spacing as calculated using FEM;

FIG. 3H is a diagram of the energy fraction (mean±sd) transferred (utilized) to the car as measured experimentally from the difference between the impact energies transmitted across the backing and the ear+backing to an underlying force sensor. FEM=finite-element modeling, exp=experiment, inf=infinite;

FIG. 4A is a schematic of a model used to simulate projection array penetration into skin backed by soft tissue; mouse car layers were modeled using an axisymmetric FE geometry with a symmetric boundary; the soft backing material was modeled using a parallel spring-damper-mass lumped element;

FIG. 4B is a schematic of the penetration depth resulting from standard treatment, i.e. firing the array with an energy of ˜13 mJ (˜35 g piston at ˜0.85 m s⁻¹) on a PDMS-backed car (left), and ˜1.3 mJ (˜5 g at ˜0.75 m s-1) on ear alone; a +15% correction factor was considered to account for the tissue shrinking due to histology treatment; the mean±se (n=4) is represented for the experimental groups, whereas the error-bars of the model group represent the uncertainty due to FE parameterization as in FIG. 4C;

FIG. 4C is a schematic of the sensitivity of the numerical solution to model parameterization when the standard treatment condition (35 g, ˜0.85 m s⁻¹) is used; the bars indicate the penetration depth resulting varying the model parameters; the direction of the depth change when the specific model parameter increases is indicated by the black curves;

FIG. 4D is a schematic of the numerical and experimental variations of penetration depth; the depth range originating from the skin variability has been represented using the deviation of the penetration measurements across biological repeats, and compared to the widest numerical variability deriving from skin properties, i.e. skin stiffness;

FIGS. 5A-5E are plots of numerical (FEM result±FE error) and experimental (exp mean±se) penetration depths as a function of varying application conditions and array designs: FIG. 5A is a plot of penetration depth versus application velocity; FIG. 5B is a plot of penetration depth versus piston mass; FIG. 5C is a plot of penetration depth versus array size; FIG. 5D is a plot of penetration depth versus projection density; FIG. 5E is a plot of penetration depth versus energy/projection, in which the significant Spearman correlation (p<0.0001) found between penetration depth pd and application energy per projection U was modeled with power laws pd=A U^{B}, i.e. straight (dotted) lines in Log-Log scale, horizontal error-bars represent the standard deviation of the measurement of application velocity and number of microprojections on the array following wafer dicing, and vertical error-bars were obtained as in FIG. 4B;

FIG. 5F is a schematic representation of applicator function and main parameters;

FIG. 6A is a plot of penetration depth versus piston mass under conditions where 1) constant spring load; 2) constant energy and 3) constant velocity;

FIG. 6B is a plot of penetration depth versus array size under conditions where 1) constant projection density and 2) constant projection number;

FIG. 6C is a plot of penetration depth versus energy/projection comparing experimental and FEM for velocity sets, mass sets, array-size-sets and density sets, in which the penetration depth escapes the Log-Log linear dependence with application energy per projection for very low piston masses and large array sizes; error-bars were omitted for clarity;

FIG. 6D is a plot of the percentage of application energy transferred to the skin versus piston mass;

FIG. 6E is a plot of the percentage of application energy transferred to the skin versus array size;

FIG. 7 is a flowchart of skin failure model, in which the clockwise flow describes the approach used in the present application; whereas the anti-clockwise flow (in grey) shows the simplified implementation used in previous work (VM=von Mises);

FIG. 8 is a plot of force measured by piezoelectric load cell placed under the PDMS following ˜2 ms⁻¹ impact of a microprojection array on the PDMS-backed skin (‘PDMS+ear’) and flat patch on PDMS backing only (‘PDMS’);

FIG. 9 is a plot of force versus compression displacement for impact tests that were performed on carbon tab-topped PDMS firing a 5 mm-diameter flat-ended piston; piston mass and impact velocity (and relative theoretical peak engineering strain rate) are indicated; the green datasets have ˜constant kinetic energy; the vertical error-bars indicate the sd of the measurements across the different PDMS samples; the horizontal error-bars of the impact tests show the uncertainty (sd over the different PDMS samples) of the compression displacement measures using the high-speed camera; and the full and dashed lines show the stiffness curves selected after PDMS model validation for the brass and plastic pistons, respectively.

FIG. 10 is a schematic diagram of model geometry of uncoated (full) and coated (dashed) microprojection.

FIGS. 11A and 11B are a schematic representations of an applicator that includes a housing containing a driver, a tubular spacer that in use is positioned with an open end in contact with a surface of skin to thereby space the housing from the skin and a stop positioned proximate the open end of the spacer. The driver drives a microprojection array releaseably mounted to the driver and the microprojection array is released from the driver prior to the microprojection array contacting the car.

### DETAILED DESCRIPTION OF THE PREFERRED EMBODIMENTS

In-depth understanding of skin elastic and rupture behaviors is important for next-generation biomedical devices because it enables targeted delivery of vaccines, as well as minimally-invasive extraction of diagnostic biomarkers and robotic/haptic surgery. Penetration of the skin's superficial barriers and precise targeting of strata rich in antigen-presenting cells is critical to elicit potent low-dose immunogenicity. However, the paucity of relevant skin mechanical characterization and lack of established fracture models has limited the rational design of cutaneous devices. The present invention exploits experimental and numerical studies of skin mechanics during dynamic interaction with individual and arrays of microscopic penetrators to provide improved methods and devices for delivering active agents into the skin. Micro-indentation of individual strata reveals that the hyperelastic moduli are dramatically rate-dependent, and allows extrapolation of the stiffness properties at velocity regimes (>mm s⁻¹) relevant for dynamically-actuated cutaneous devices. These are used to parameterize a layered finite-element (FE) representation of skin that includes a novel implementation of ductile failure. Iterative refinement to match empirical penetration assays yields characteristic fracture energies (˜10 pJ μm⁻²) significantly lower than previously reported (>>100 pJ μm⁻²). The resulting FE simulations satisfactorily predict the penetration depth of microprojection arrays across a diverse range of designs and application conditions, and shows limited sensitivity to the parameterization choice. The knowledge and numerical tools developed provide guidelines to rationally engineer skin penetrators. Specific array design and application conditions can be developed to increase the targeting consistency across patients and minimize the penetration energy while controlling skin inflammation, tolerability and acceptability.

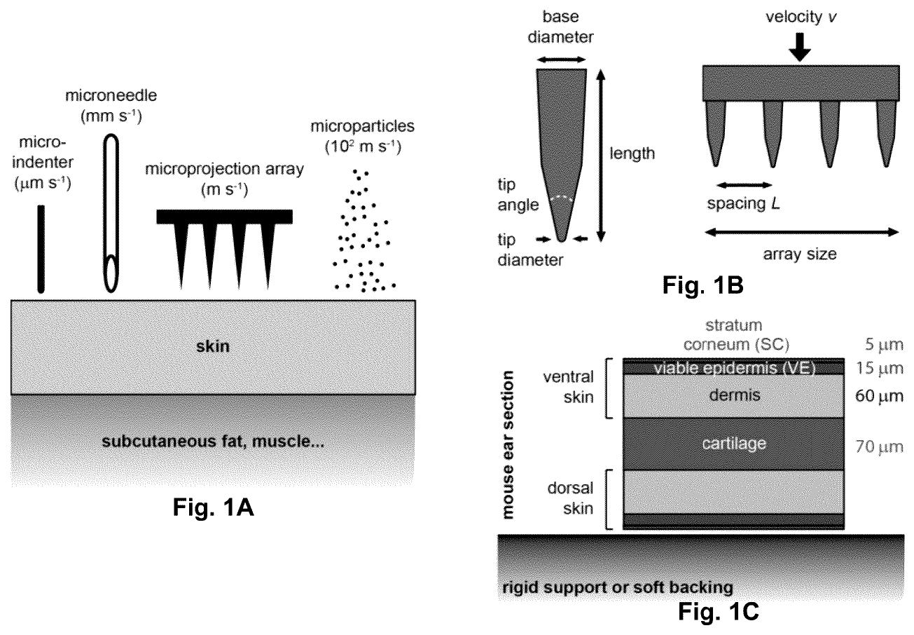

Both experiments and theoretical models were used to develop an understanding of the skin's mechanical properties relative to the dynamic penetration of individual and multiple microscopic penetrators. These properties are particularly relevant to the skin treatment by microneedles/microprojections for vaccine delivery as well as minimally-invasive extraction of diagnostic biomarkers. Starting from micro-indentation experiments on mouse skin (FIG. 1C), the hyperelastic properties of the epidermal and dermal layers at high strain-rates (>1 μs⁻¹) were derived. These were utilized in conjunction with finite-element simulations to further investigate the rate-dependent skin mechanical response to the impact of individual and arrays of penetrator tips.

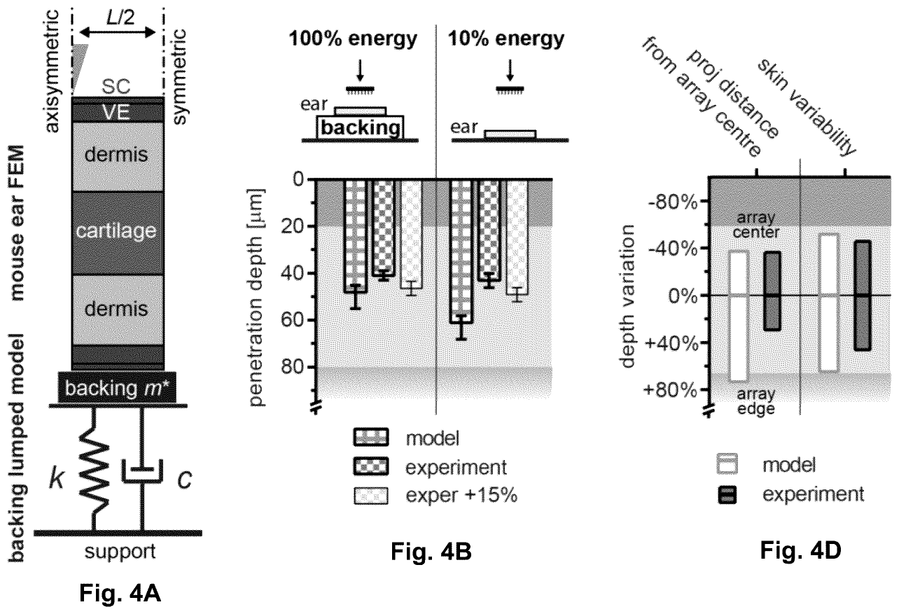

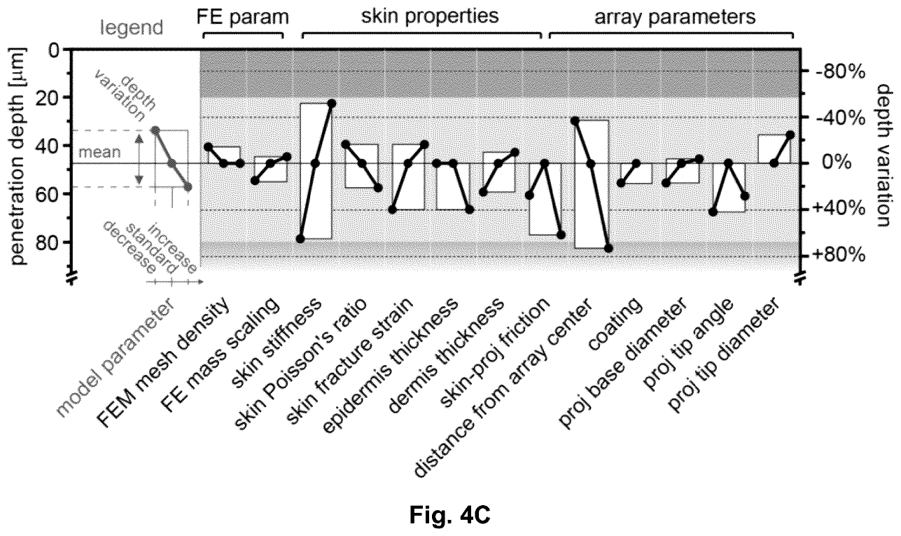

The complete model schematized in FIG. 4A was used to simulate skin mechanical interaction with the microprojections in the conditions used for mouse vaccination experiment (G. J. P. Fernando, X. F. Chen, T. W. Prow, M. L. Crichton, E. J. Fairmaid, M. S. Roberts, I. H. Frazer, L. E. Brown, M. A. F. Kendall, PLoS One 2010, 5, e10266). Penetration was studied for varying array designs and application parameters. For validation, the calculated penetration depths were compared with experimental measurements from histological sections of skin treated with dye-coated arrays according to an established protocol (M. L. Crichton, A. Ansaldo, X. F. Chen, T. W. Prow, G. J. P. Fernando, M. A. F. Kendall, Biomaterials 2010, 31, 4562).

FIG. 4B shows the simulation and experimental results for a 4×4 mm² array containing ˜3000 microprojections spaced of L=70 μm (i.e. ˜20 kproj cm⁻²) applied on PDMS-backed skin at 0.85 m s⁻¹ with the 35 g piston (i.e. ˜13 mJ), the ‘standard treatment’ condition. The resulting penetration depth, 48 μm from the model, is in good agreement with the experimental measurement, 41±2 μm (mean±se). This simulation indicated that 6.2% of the energy is transferred to the skin. The model was revised by removing the backing and applying the array to the ear alone using (conservatively) ˜10% of the energy (˜5 g at ˜0.75 m s⁻¹, i.e. ˜1.3 mJ). FIG. 4B shows that this reduced-energy condition penetrates un-backed skin to a depth comparable with the standard treatment on backed skin, which further validates the skin and PDMS parameterizations.

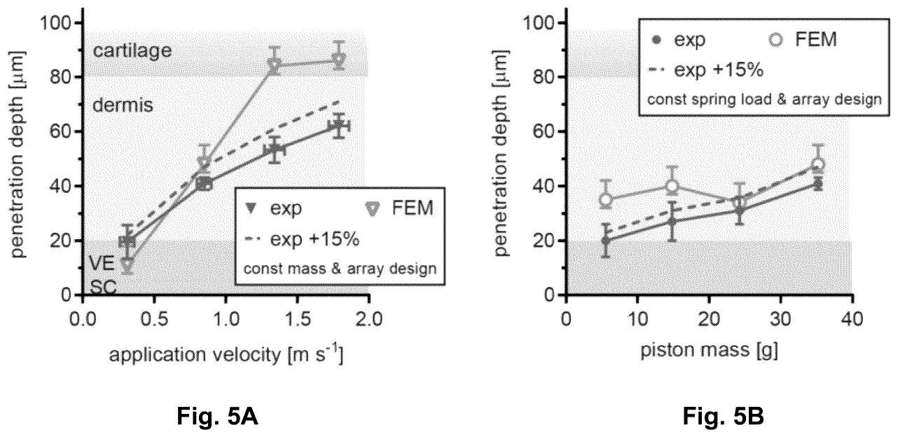

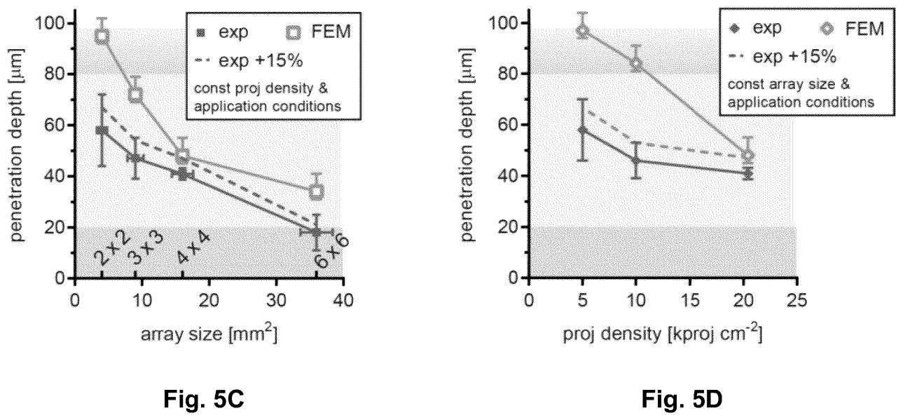

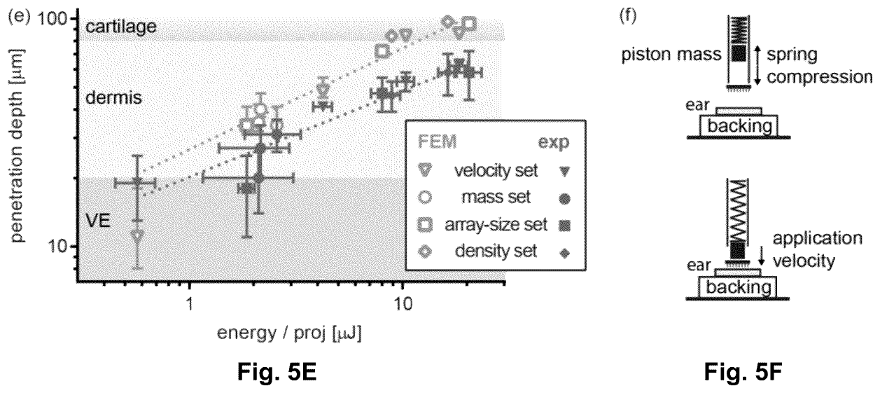

The sensitivity of the numerically-derived penetration depth to the variation of the model parameters was assessed with a set of limit analyses. In brief, the standard treatment simulation was repeated assigning upper and lower boundary values to each individual parameter, one at a time. The input-parameter intervals are summarized in Table 1 and are representative of the range of FE parameters, variation of skin properties as reported in the literature and possible array design tolerances or modifications. For simple reference to FIGS. 5A-5E, the top, respectively bottom, row in Table 1 shows the condition resulting into shallower, respectively deeper, penetration.

TABLE 1
Summary of parameter variation ranges used to assess the sensitivity of the numerical solutions.

| | FE | | | | Skin | Skin | | | | Array | Array | Array |
|---|---|---|---|---|---|---|---|---|---|---|---|---|
| FE | mass | Skin | Skin | Skin | epider | dermis | Skin- | Array | | proj | proj tip | proj tip |
| mesh | scaling | elastic | Poisson's | fracture | thickn | thickn | proj | proj | Array | diamet | angle | diamet |
| density | [ps] | moduli | ratio | strain | [μm] | [μm] | friction | location | coating | [μm] | [deg] | [μm] |
| −50% | ~100 | +70% | 0.35 | >20% | 9 | 80 | | center | | 29 | | 5 |
| st^{a)} | ~50 | st^{b)} | 0.45 | ≤20%^{c)} | 20 | 60 | 0.4 | mean^{d)} | yes^{e)} | 23 | 30 | 1 |
| +25%. | | | | | | | | | | | | |
| | none | −50% | 0.49 | 0% | 27 | 40 | 0.7 | edge | no | 17 | 50 | |
| | | | | | | | 0.05 | | | | 10 | |

st = standard value.

FIG. 4C shows that no significant difference resulted after refining the mesh, which indicates that the mesh of choice is appropriate. Among other skin characteristics, penetration depth was most sensitive to strata stiffness (FIG. 4C); and, interestingly, the resulting numerical depth range is in close agreement with the measurement variation across biological repeats (FIG. 4D). On the other hand, the experiments revealed a significantly deeper penetration depth towards the edges of the array, likely due to the larger force exerted by peripheral microprojections. By scaling the microprojection momentum the increasing penetration depth caused by projections located at increasing distance from the array center could be reasonably predicted (FIG. 4D).

The penetration resulting from different array application conditions (FIGS. 5A-5B) and designs (FIGS. 5C-5D) was investigated numerically and empirically by further applying the computational and experimental methods. Increasing microprojection velocity resulted in deeper penetration due to the larger energy. Separately, lower piston masses (using the same application spring load) resulted in slightly decreasing penetration, despite the theoretically-constant application potential energy. In fact, applicator characterization revealed lower than expected application velocities for the lower masses (<35 g), possibly due to a greater friction of the lighter plastic piston against the applicator housing compared to the standard brass piston. The simulations were run using the measured velocities, rather than the theoretically-calculated ones. Decreasing the array size or the microprojection density (constant array size) resulted in deeper penetration mostly because the same application energy is shared among fewer projections. The numerical prediction and the experimental measurement were in reasonable agreement. Specifically, the model appears to overestimate the depth especially when the projections are widely spaced and approach the deep dermis. This is possibly due to two reasons: 1) the deeper penetration of the peripheral projections (FIG. 4D) might allow contact between the SC and the base of the array, especially for sparse arrays; and 2) the projection interacts with the cartilage, which mechanical properties were not accurately established.

There is significant Spearman correlation (p<0.0001) between the penetration depth pd and the application energy per projection U (FIG. 5E). The power (1.30±0.04) U^{(0.38±0.04)} (mean±se) fitted the experimental data satisfactorily (R²=0.931). An analogous non-linear regression for the numerical dataset yielded (1.43±0.05) U^{(0.44±0.05)} with similar goodness-of-fit (R²=0.932). These curves pd=A U^{B} appear as straight lines in Log-Log scale (FIG. 5E) where A is the intercept, B is the slope, the depth pd is measured in μm and U in μJ. FIG. 5E also suggests that the penetration depth of arrays with custom design and application conditions can be simplistically predicted from the application energy (per projection) using this empirical relationship.

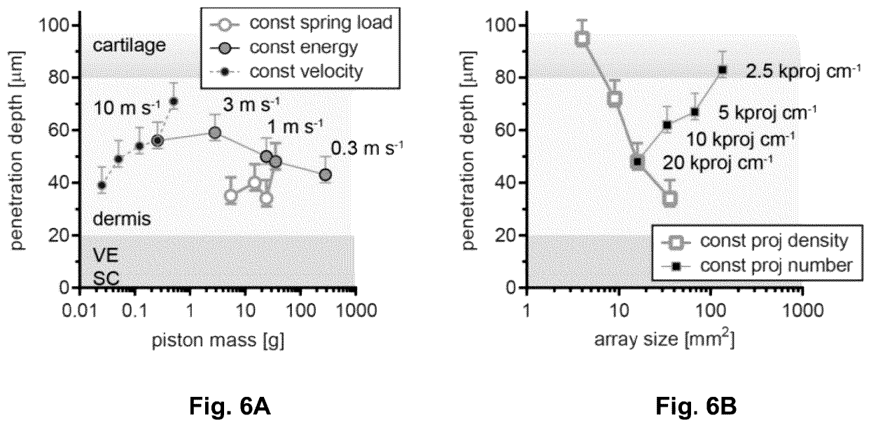

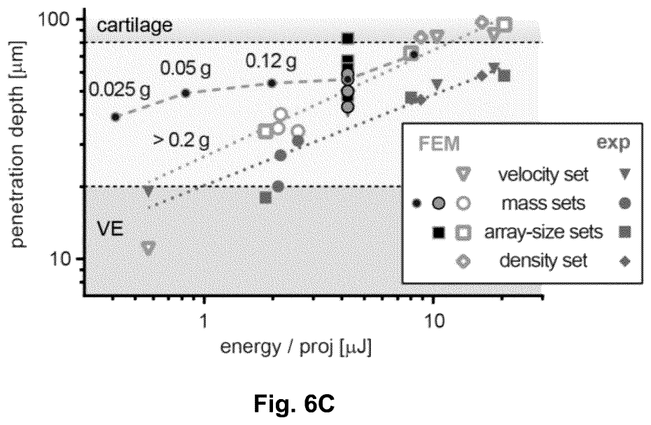

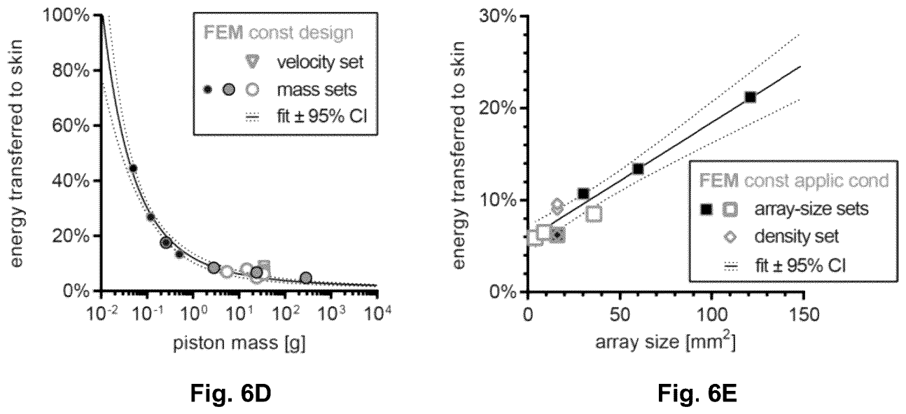

The computational model was applied to investigate alternative designs and application conditions and challenge the trend of FIG. 5E. Interestingly, decreasing piston mass (FIG. 6A) or increasing the array size (FIG. 6B) resulted in increased penetration depth although the energy per projection was held constant. These conditions, as well as 10 m s⁻¹ applications for masses below 0.2 g (FIG. 6A) markedly violated the Log-Log linear relationship between depth and energy per projection (FIG. 6C). Specifically, the results indicate that isoenergetic applications achieve a ˜2-fold deeper penetration using a mass <0.05 g or spreading the microprojections over a 10-fold larger area. Equivalently, the energy required to reach a mid-dermal depth (˜50 μm) can be reduced by over 80% by lowering the mass from 35 g to 0.05 g. Key for this ‘energy sparing’ phenomenon is the increasing application velocity required to maintain a constant energy while decreasing the mass. In fact, the simulations of velocities <3 m s⁻¹ showed that skin fracture starts after a large compression of the backing and terminates after 0.5-1 ms. In contrast, the fracture process is completed in ˜10 μs at 10 m s⁻¹, before the backing has started to deform. Likely, these different penetration regimes arise because the projection motion competes with the transmission of the deformation to the backing through the stress waves. Such behavior suggests that an efficiency around 55% can be theoretically achieved by reducing the moving mass down to the array itself (˜0.03 g). In addition, the energy transfer efficiency linearly correlated with array size (FIG. 6E; Pearson's r=0.966, p<0.0001, slope=(0.126+0.013)% mm⁻², intercept=(5.78±0.62) %). This is likely to be because distributing the impact over a larger surface increases the overall backing elastic force response, thus results in an effectively stiffer substrate.

The results of FIGS. 6A-6E indicate that penetration depth is not a unique function of the energy per projection. Rather, the application energy required to target a specific depth can be modulated by varying the velocity-to-mass ratio. This represents an important degree of freedom to seek immunologically-beneficial levels of inflammation (e.g. cell stress/death via mechanical perturbation) without compromising treatment tolerability and acceptability by the patient. On the other hand, high-velocity, low-mass applications allow the microprojections to interact mainly with the superficial layers (i.e. the skin). This effectively reduces the dependence of penetration on the skin backing properties, hence potentially improves the targeting consistency across patients with different subcutaneous tissue composition (e.g. different body-mass index).

The skin dynamic behavior is the main cause of such a diverse mechanical response. Firstly, the heterogeneous layered composition favored fracture in the early impact stages for large application velocities. Specifically, the stress was effectively retained at the surface due to the slow stress-wave propagation of the deep strata (cartilage, PDMS, fat or muscle), comparatively lower in stiffness. Secondly, the equivalent strain required to initiate failure (i.e. meet the yield criterion) decreased with increasing velocity because skin elasticity (i.e. the stress response to a specific strain) has a steeper rate-dependent increase compared to the yield strength. As a consequence, penetration is more difficult in quasi-static conditions, as the Young's modulus-to-yield strength ratio decreases below 1, due to the resulting strata softness (compliance).

The resulting penetration model satisfactorily reproduced the experimental behavior for a wide range of conditions, and further proved robust to variations in parameterization. However, the utilized elastic moduli were derived from indentations using constant probe velocity, and are relative to the peak strain rates at impact. Hypothetically, the resulting skin stress relaxation should result in lower penetration depths that match the experimental measurements more closely.

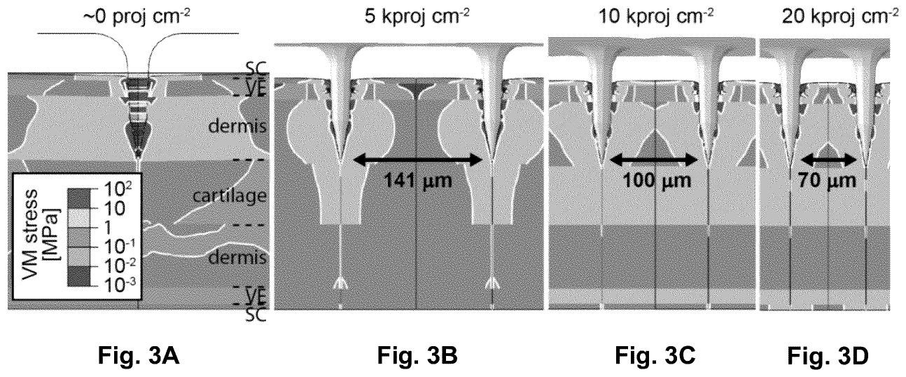

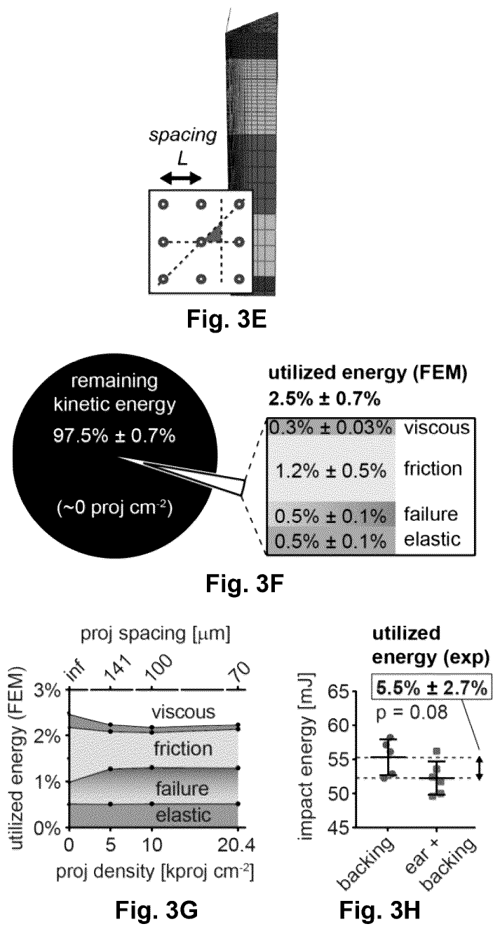

While significant differences in skin behavior are expected if the dynamic regime is changed (e.g. from impact to quasi-static or vibratory application), penetration of other microneedle array designs (typically characterized by sparser, larger penetrators) will likely follow the trends showed in FIGS. 5A-5E and FIGS. 6A-6E. This is justified by the low variation between the relative energetic contributions (e.g. fracture, deformation and friction) (FIG. 3G) and the approximately constant stress generated as tip radius and spacing increase. As can be seen in FIGS. 4A-4D a variety of parameters may affect the depth of penetration of microprojections into the skin: skin stiffness, skin fracture strain, epidermis thickness, dermis thickness, skin-microprojection friction, distance of projections from the array center, amount of coating on microprojection, microprojection tip angle, microprojection shape, velocity of microprojection array into the skin, mass of microprojection array, velocity to mass ratio of the microprojection area, area of the microprojection array, density of microprojection array, backing used behind skin target.

When administered to the skin the microprojection array may have a velocity which is greater than about 5 m/s or about 6 m/s, or about 7 m/s, or about 8 m/s, or about 9 m/s, or about 10 m/s, or about 15 m/s, or about 20 m/s, or about 25 m/s, or about 30 m/s, or about 40 m/s, or about 45 m/s, or about 50 m/s, or about 55 m/s. When administered to the skin the microprojection array may have a velocity which is about 5 m/s to about 50 m/s, or from about 5 m/s to about 45 m/s, or from 5 m/s to about 40 m/s, or from about 5 m/s to about 35 m/s, or from about 5 m/s to about 30 m/s, or from 5 m/s to about 25 m/s, or from about 5 m/s to about 20 m/s, or from about 5 m/s to about 15 m/s, or from 5 m/s to about 10 m/s, or from about 10 m/s to about 50 m/s, or from about 10 m/s to about 45 m/s, or from 10 m/s to about 40 m/s, or from about 10 m/s to about 35 m/s, or from about 10 m/s to about 30 m/s, or from 10 m/s to about 25 m/s, or from about 10 m/s to about 20 m/s, or from about 10 m/s to about 15 m/s, or from about 15 m/s to about 50 m/s, or from about 15 m/s to about 45 m/s, or from 15 m/s to about 40 m/s, or from about 15 m/s to about 35 m/s, or from about 15 m/s to about 30 m/s, or from 15 m/s to about 25 m/s, or from about 15 m/s to about 20 m/s, or from about 20 m/s to about 50 m/s, or from 20 m/s to about 45 m/s, or from about, or from 20 m/s to about 40 m/s, or from about 20 m/s to about 35 m/s, or from about 20 m/s to about 30 m/s, or from about 20 m/s to about 25 m/s, or from about 25 m/s to about 50 m/s, or from about 25 m/s to about 45 m/s, or from 25 m/s to about 40 m/s, or from about 25 m/s to about 35 m/s, or from about 25 m/s to about 30 m/s, or from about 30 m/s to about 50 m/s, or from about 30 m/s to about 45 m/s, or from about 30 m/s to about 40 m/s, or from about 30 m/s to about 35 m/s.

The microprojection arrays may have a mass of less than 1 gram, or less than 0.9 grams, or less than 0.8 grams, or less than 0.7 grams, or less than 0.6 grams, or less than 0.5 grams, or less than 0.6 grams, or less than 0.5 grams, or less than 0.4 grams, or less than 0.3 grams, or less than 0.2 grams, or less than 0.1 grams, or less than 0.05 grams, or less than 0.01 grams, or less than 0.005 grams, or less than 0.001 grams. The microprojection array may have a mass of from about 0.001 grams to about 5 grams of about 0.001 grams to about 2 grams, or from about 0.001 grams to about 1.5 grams, or from about 0.001 grams to about 1.0 grams, or from about 0.001 grams to about 0.9 grams, or from about 0.001 grams to about 0.8 grams, or from about 0.001 grams to about 0.7 grams, or from about 0.001 grams to about 0.6 grams, or from about 0.001 grams to about 0.5 grams, or from about 0.001 grams to about 0.4 grams, or from about 0.001 grams to about 0.3 grams, or from about 0.001 grams to about 0.2 grams, or from about 0.001 grams to about 0.1 grams from about 0.01 grams to about 5 grams of about 0.01 grams to about 2 grams, or from about 0.01 grams to about 1.5 grams, or from about 0.01 grams to about 1.0 grams, or from about 0.01 grams to about 0.9 grams, or from about 0.01 grams to about 0.8 grams, or from about 0.01 grams to about 0.7 grams, or from about 0.01 grams to about 0.6 grams, or from about 0.01 grams to about 0.5 grams, or from about 0.01 grams to about 0.4 grams, or from about 0.01 grams to about 0.3 grams, or from about 0.01 grams to about 0.2 grams, or from about 0.01 grams to about 0.1 grams, or from about 0.05 grams to about 5 grams of about 0.05 grams to about 2 grams, or from about 0.05 grams to about 1.5 grams, or from about 0.05 grams to about 1.0 grams, or from about 0.05 grams to about 0.9 grams, or from about 0.05 grams to about 0.8 grams, or from about 0.05 grams to about 0.7 grams, or from about 0.05 grams to about 0.6 grams, or from about 0.05 grams to about 0.5 grams, or from about 0.05 grams to about 0.4 grams, or from about 0.05 grams to about 0.3 grams, or from about 0.05 grams to about 0.2 grams, or from about 0.05 grams to about 0.1 grams, or from about 0.1 grams to about 1.0 grams, or from about 0.1 grams to about 5 grams, or from about 0.1 grams to about 2 grams, or from about 0.1 grams to about 0.9 grams, or from about 0.1 grams to about 0.8 grams, or from about 0.1 grams to about 0.7 grams, or from about 0.1 grams to about 0.6 grams, or from about 0.1 grams to about 0.5 grams, or from about 0.1 grams to about 0.4 grams, or from about 0.1 grams to about 0.3 grams, or from about 0.1 grams to about 0.2 grams.

The density of the microprojection on the microprojection arrays may be about 2000microprojections/cm², or about 2500 microprojections/cm², or about 3000 microprojections/cm², or about 3500 microprojections/cm², or about 4000 microprojections/cm², or about 4500 microprojections/cm², or about 5000 microprojections/cm², or about 5500 microprojections/cm², or about 6000 microprojections/cm², or about 6500 microprojections/cm², or about 7000 microprojections/cm², or about 7500 microprojections/cm², or about 8000 microprojections/cm², or about 8500 microprojections/cm², or about 9000 microprojections/cm², or about 9500 microprojections/cm², or about 10000 microprojections/cm², or about 11000 microprojections/cm², or about 12000 microprojections/cm², or about 13000 microprojections/cm², or about 14000 microprojections/cm², or about 15000 microprojections/cm², or about 16000 microprojections/cm², or about 17000 microprojections/cm², or about 18000 microprojections/cm², or about 19000 microprojections/cm², or about 20000 microprojections/cm². The density of the microprojection on the microprojection arrays may be from about 2000 to about 20000 microprojections/cm², or from about 2000 to about 15000 microprojections/cm², or from about to about 10000 microprojections/cm², or from about 2000 to about 9000 microprojections/cm², or from about 2000 to about 8000 microprojections/cm², or from about 2000 to about 7500 microprojections/cm², or from about 2000 to about 7000 microprojections/cm², or from about 2000 to about 6000 microprojections/cm², or from about 2000 to about 5000 microprojections/cm², or from about 2000 to about 4000 microprojections/cm², or from about 3000 to about 20000 microprojections/cm², or from about 3000 to about 15000 microprojections/cm², or from about to about 10000 microprojections/cm², or from about 3000 to about 9000 microprojections/cm², or from about 3000 to about 8000 microprojections/cm², or from about 3000 to about 7500 microprojections/cm², or from about 3000 to about 7000 microprojections/cm², or from about 3000 to about 6000 microprojections/cm², or from about 3000 to about 5000 microprojections/cm², or from about 3000 to about 4000 microprojections/cm², or from about 4000 to about 20000 microprojections/cm², or from about 4000 to about 15000 microprojections/cm², or from about to about 10000 microprojections/cm², or from about 4000 to about 9000 microprojections/cm², or from about 4000 to about 8000 microprojections/cm², or from about 4000 to about 7500 microprojections/cm², or from about 4000 to about 7000 microprojections/cm², or from about 4000 to about 6000 microprojections/cm², or from about 4000 to about 5000 microprojections/cm², or from about 5000 to about 20000 microprojections/cm², or from about 5000 to about 15000 microprojections/cm², or from about to about 10000 microprojections/cm², or from about 5000 to about 9000 microprojections/cm², or from about 5000 to about 8000 microprojections/cm², or from about 5000 to about 7500 microprojections/cm², or from about 5000 to about 7000 microprojections/cm², or from about 5000 to about 6000 microprojections/cm².

At least a portion of the projections may be coated. Accordingly, one way of providing material for delivery to the biological subject is by providing the material within the coating. For example, the coating may include a vaccine for providing an immunological response within the subject. The coating may be provided in liquid or non-liquid forms, and may further include ingredients other than the material to be delivered, such as an adjuvant. Suitable coating formulations for use with projections patches and methods of applying such coatings to the projections are known, as described, for example, in WO/2010/042996 and WO/2009/079712.

Although any type of coating may be used, particularly advantageous embodiments of the microprojection arrays are provided with at least a portion of the projections coated with a non-liquid coating. In this regard, the term “non-liquid” coating will be understood to include a coating that is applied in a liquid form and allowed to dry or otherwise solidify to thereby form a non-liquid coating.

The non-liquid coating may act as an additional substantially solid layer of material which can be used to even further adjust the geometry of the projections by optionally causing the projections to have an effective profile of a different shape to the underlying uncoated profile of the projections as initially fabricated.

The microprojections of the array of the present invention may be of any shape including cylindrical or conical. Other geometries are also possible. The microprojection arrays may have substrate with a plurality of microprojections protruding from the substrate wherein the microprojections have a tapering hexagonal shape and comprise a tip and a base wherein the base has two substantially parallel sides with a slight draught angle of approximately 1 to 20 degrees up to a transition point at which point the angle increases to from about 20 degrees to about 70 degrees. A sharp blade-like tip will allow for enhanced penetration of the microprojections into the skin while also generating an enhanced localized cell death/bystander interaction in the skin with a different profile than conical microprojection arrays. The sharp blade-like tips of the microprojections may also increase the level of danger signals and antigen to more live cells thereby increasing the physical adjuvant effect of microprojections and thereby improving immune responses. The tip of the microprojections of the present invention may have a width of about 0.5 μm, or about 1.0 μm, or about 1.5 μm, or about 2.0 μm, or about 2.5 μm, or about 3.0 μm, or about 3.5 μm, or about 4.0 μm, or about 4.5 μm, or about 5.0 μm. The tip of the microprojections of the present invention may have a width of from about 0.5 μm to about 5.0 μm, or from about 0.5 μm to about 4.5 μm, or from about 0.5 μm to about 4.0 μm, or from about 0.5 μm to about 3.5 μm, or from about 0.5 μm to about 3.0 μm, or from about 0.5 μm to about 2.5 μm, or from about 0.5 μm to about 2.0 μm, or from about 0.5 μm to about 1.5 μm, or from about 0.5 μm to about 1.0 μm, or from about 1.0 μm to about 5.0 μm, or from about 1.0 μm to about 4.5 μm, or from about 1.0 μm to about 4.0 μm, or from about 1.0 μm to about 3.5 μm, or from about 1.0 μm to about 3.0 μm, or from about 1.0 μm to about 2.5 μm, or from about 1.0 μm to about 2.0 μm, or from about 1.0 μm to about 1.5 μm, or from about 1.5 μm to about 5.0 μm, or from about 1.5 μm to about 4.5 μm, or from about 1.5 μm to about 4.0 μm, or from about 1.5 μm to about 3.5 μm, or from about 1.5 μm to about 3.0 μm, or from about 1.5 μm to about 2.5 μm, or from about 1.5 μm to about 2.0 μm, or from about 2.0 μm to about 5.0 μm, or from about 2.0 μm to about 4.5 μm, or from about 2.0 μm to about 4.0 μm, or from about 2.0 μm to about 3.5 μm, or from about 2.0 μm to about 3.0 μm, or from about 2.0 μm to about 2.5 μm, or from about 2.5 μm to about 5.0 μm, or from about 2.5 μm to about 4.5 μm, or from about 2.5 μm to about 4.0 μm, or from about 2.5 μm to about 3.5 μm, or from about 2.5 μm to about 3.0 μm.

The microprojection array when applied to the skin may have a mass-to-velocity ratio of less than about 0.0005 g/m/s, or less than about 0.001 g/m/s/or less than about 0.002 g/m/s, or less than about 0.003 g/m/s, or less than about 0.004 g/m/s/or less than about 0.005 g/m/s, or less than about 0.006/m/s, or less than about 0.007 g/m/s/or less than about 0.008 g/m/s, or less than about 0.009 g/m/s, or less than about 0.01 g/m/s/or less than about 0.02 g/m/s, or less than about 0.03/m/s, or less than about 0.04 g/m/s/or less than about 0.05 g/m/s, or less than about 0.06 g/m/s, or less than about 0.07 g/m/s/or less than about 0.08 g/m/s, or less than about 0.09/m/s, or less than about 0.10 g/m/s/or less than about 0.20 g/m/s, or less than about 0.30 g/m/s, or less than about 0.40 g/m/s/or less than about 0.50 g/m/s. The microprojection array when applied to the skin may have a mass-to-velocity ratio of about 0.0005 g/m/s to about 0.50 g/m/s, or from about 0.0005 g/m/s to about 0.40 g/m/s, or from about 0.0005 g/m/s to about 0.30 g/m/s, or from about 0.0005 g/m/s to about 0.20 g/m/s, or from about 0.0005 g/m/s to about 0.10 g/m/s, or from about 0.0005 g/m/s to about 0.009 g/m/s, or from of about 0.0005 g/m/s to about 0.008 g/m/s, or from about 0.0005 g/m/s to about 0.007 g/m/s, or from about 0.0005 g/m/s to about 0.006 g/m/s, or from about of about 0.0005 g/m/s to about 0.005 g/m/s, or from about 0.0005 g/m/s to about 0.004 g/m/s, or from about 0.0005 g/m/s to about 0.003 g/m/s, or from about of about 0.0005 g/m/s to about 0.002 g/m/s, or from about 0.0005 g/m/s to about 0.001 g/m/s, or from about 0.001 g/m/s to about 0.50 g/m/s, or from about 0.001 g/m/s to about 0.40 g/m/s, or from about 0.001 g/m/s to about 0.30 g/m/s, or from about 0.001 g/m/s to about 0.20 g/m/s, or from about 0.001 g/m/s to about 0.10 g/m/s, or from about 0.001 g/m/s to about 0.009 g/m/s, or from of about 0.001 g/m/s to about 0.008 g/m/s, or from about 0.001 g/m/s to about 0.007 g/m/s, or from about 0.001 g/m/s to about 0.006 g/m/s, or from about of about 0.001 g/m/s to about 0.005 g/m/s, or from about 0.001 g/m/s to about 0.004 g/m/s, or from about 0.001 g/m/s to about 0.003 g/m/s, or from about of about 0.001 g/m/s to about 0.002 g/m/s, or from about 0.005 g/m/s to about 0.50 g/m/s, or from about 0.005 g/m/s to about 0.40 g/m/s, or from about 0.005 g/m/s to about 0.30 g/m/s, or from about 0.005 g/m/s to about 0.20 g/m/s, or from about 0.005 g/m/s to about 0.10 g/m/s, or from about 0.005 g/m/s to about 0.009 g/m/s, or from of about 0.005 g/m/s to about 0.008 g/m/s, or from about 0.005 g/m/s to about 0.007 g/m/s, or from about 0.005 g/m/s to about 0.006 g/m/s, or from about 0.033 g/m/s to about 0.0008 g/m/s.

The area of the microprojection arrays in area may be between about 10 mm² to about 1000 mm², or from about 10 mm² to about 900 mm², or from about 10 mm² to about 800 mm², or from about 10 mm² to about 700 mm², or from about 10 mm² to about 600 mm², or from about 10 mm² to about 600 mm², or from about 10 mm² to about 500 mm², or from about 10 mm² to about 400 mm², or from about 10 mm² to about 300 mm², or from about 10 mm² to about 200 mm², or from about 10 mm² to about 100 mm², or from about 10 mm² to about 90 mm², or from about 10 mm² to about 80 mm², or from about 10 mm² to about 70 mm², or from about 10 mm² to about 60 mm², or from about 10 mm² to about 50 mm², or from about 10 mm² to about 40 mm², or from about 10 mm² to about 30 mm², or from about 10 mm² to about 20 mm², or from about 20 mm² to about 1000 mm², or from about 20 mm² to about 900 mm², or from about 20 mm² to about 800 mm², or from about 20 mm² to about 700 mm², or from about 10 mm² to about 600 mm², or from about 20 mm² to about 500 mm², or from about 20 mm² to about 400 mm², or from about 20 mm² to about 300 mm², or from about 20 mm² to about 200 mm², or from about 20 mm² to about 100 mm², or from about 20 mm² to about 90 mm², or from about 20 mm² to about 80 mm², or from about 20 mm² to about 70 mm², or from about 20 mm² to about 60 mm², or from about 20 mm² to about 50 mm², or from about 20 mm² to about 40 mm², or from about 20 mm² to about 30 mm², or from about 30 mm² to about 1000 mm², or from about 30 mm² to about 900 mm², or from about 30 mm² to about 800 mm², or from about 30 mm² to about 700 mm², or from about 10 mm² to about 600 mm², or from about 30 mm² to about 500 mm², or from about 30 mm² to about 400 mm², or from about 30 mm² to about 300 mm², or from about 30 mm² to about 200 mm², or from about 30 mm² to about 100 mm², or from about 30 mm² to about 90 mm², or from about 30 mm² to about 80 mm², or from about 30 mm² to about 70 mm², or from about 30 mm² to about 60 mm², or from about 30 mm² to about 50 mm², or from about 30 mm² to about 40 mm², or from about 40 mm² to about 1000 mm², or from about 40 mm² to about 900 mm², or from about 40 mm² to about 800 mm², or from about 40 mm² to about 700 mm², or from about 10 mm² to about 600 mm², or from about 40 mm² to about 500 mm², or from about 40 mm² to about 400 mm², or from about 40 mm² to about 400 mm², or from about 40 mm² to about 200 mm², or from about 40 mm² to about 100 mm², or from about 40 mm² to about 90 mm², or from about 40 mm² to about 80 mm², or from about 40 mm² to about 70 mm², or from about 40 mm² to about 60 mm², or from about 40 mm² to about 50 mm², or from about 50 mm² to about 1000 mm², or from about 50 mm² to about 900 mm², or from about 50 mm² to about 800 mm², or from about 50 mm² to about 700 mm², or from about 10 mm² to about 600 mm², or from about 50 mm² to about 500 mm², or from about 50 mm² to about 400 mm², or from about 50 mm² to about 300 mm², or from about 50 mm² to about 200 mm², or from about 50 mm² to about 100 mm², or from about 50 mm² to about 90 mm², or from about 50 mm² to about 80 mm², or from about 50 mm² to about 70 mm², or from about 50 mm² to about 60 mm², or from 60 mm² to about 1000 mm², or from about 60 mm² to about 900 mm², or from about 60 mm² to about 800 mm², or from about 60 mm² to about 700 mm², or from about 10 mm² to about 600 mm², or from about 60 mm² to about 500 mm², or from about 60 mm² to about 400 mm², or from about 60 mm² to about 300 mm², or from about 60 mm² to about 600 mm², or from about 60 mm² to about 100 mm², or from about 60 mm² to about 90 mm², or from about 60 mm² to about 80 mm², or from about 60 mm² to about 70 mm², or from about 16 mm² to about 400 mm², or from about 36 mm² to about 225 mm², or from about 64 mm² to about 100 mm².

The microprojections of the microprojection arrays of the present invention may be solid or non-porous or contain hollow portions therein. In some embodiments the microprojection as solid and non-porous and do not contain hollow portion therein. In preferred embodiments the devices of the present invention do not contain reservoirs.

In view of the above, it will be appreciated that the present invention is generally directed to devices and methods for intradermal delivery of active agents into the skin. The invention is directed to devices and methods for improving the immunogenicity of vaccine preparations by intradermal delivery of the vaccine via a microprojection array in which the parameters for delivery of the active agents have been developed to achieve appropriate depth penetration and efficient delivery of the active agent.

The methods of the present invention may be used to design vaccination devices as well as develop the parameters for delivery of vaccines efficiently and minimize the penetration energy of the array while controlling skin inflammation, tolerability and acceptability. The present methods further enable investigation of the application of other cutaneous devices (e.g. solid, hollow, or dissolvable penetrators of custom size, possibly arranged in linear, rectangular or round arrays of arbitrary density) to different skin types.

The present invention relates to microprojection arrays wherein the physical parameters of the arrays such as but not limited to array mass, microprojection density, microprojection diameter, array size, microprojection tip angle, microprojection base diameter are determined for a given application.

The present invention relates to microprojection arrays wherein the physical parameters of the arrays can be determined for a given penetration depth range.

The present invention relates to methods of designing the physical parameters of microprojection arrays for a given penetration depth range.

### EXAMPLES

### Example 1

### Microprojection Array Application to Mouse Skin

Microprojection arrays were fabricated using a deep-reactive ion etching approach and diced from silicon wafers by the Australian National Fabrication Facility (ANFF) at The University of Queensland as previously described (D. Jenkins, S. Corrie, C. Flaim, M. Kendall, RSC Advances 2012, 2, 3490). Arrays were first cleaned in 70% ethanol for 10 min, flushed with an excess of water, then dried under ambient conditions. Prior application to skin, the arrays were coated with fluorescent nanoparticles (Fluospheres®, 0.2 mm, Yellow Green Fluorescent 505/515 nm, 2% Solids, Molecular Probes®, Oregon, USA) as described by Coffey et al (J. W. Coffey, S. R. Corrie, M. A. Kendall, Biomaterials 2013, 34, 9572). In brief, 8 μL of solution containing Fluospheres® with 0.2% solids and 1% methylcellulose (w/v methylcellulose, Sigma-Aldrich, USA) was deposited onto a 4×4 mm² array and dried using a rotating nitrogen jet to evenly distribute the solution on the whole array while simultaneously localizing the respective payload on the projection (X. Chen, T. W. Prow, M. L. Crichton, D. W. Jenkins, M. S. Roberts, I. H. Frazer, G. J. Fernando, M. A. Kendall, J Control Release 2009, 139, 212). The volume was 4.5 μL and 18 μL for the 3×3 mm² and 6×6 mm² arrays, respectively, to maintain a constant coating volume per unit array area. Coated arrays were stored in sealed Petri dishes protected from light until used. Scanning electron Microscopy (SEM) was performed before and after coating to ensure microprojection integrity and shape consistency. The arrays selected measured (uncoated) 90-110 μm in length, 16-20 μm in width at the base, and tapered a 15°-25° angle terminating in a tip of ˜1 μm in diameter. Coating increased base width increase of ˜4 μm and the tip angle to ˜35°. Female BALB/c mice aged 6 to 8 weeks were chosen because commonly used for immunology experiments and due to the reduced speckling during tissue imaging. The mice were anesthetised prior to array application with a solution of 60 μL of 25 mg/mL ketamine and 5 mg/mL xylazine in saline via intraperitoneal injection and were treated according to the protocol approved by the University of Queensland Animal Ethics Committee. Arrays were applied to the inner earlobe of the ears using an applicator device consisting of a sprung piston. Different impact velocities and energies were generated firing pistons of different masses and varying the initial spring compression through holes drilled in the cylinder housing. The mass was decreased from the standard 35 g of the brass piston, using a plastic piston jointly with ˜9 g incremental weights screwed on its top end. During application, the car rested on a 3 mm-PDMS backing slab. Adhesive carbon tabs fixed the car to the PDMS and the PDMS to the bench support. The array was left in place for 2 min and then carefully removed. The animals were euthanized immediately after treatment through cervical dislocation and the cars excised for experimental characterization.

### Example 2

### Experimental Characterization of Skin Penetration

The excised car specimen was immediately fixed by immersion into in 2% paraformaldehyde in phosphate buffer saline (PBS) for ˜2 hours, and then frozen in Optimal Cutting Temperature® (OCT) compound (Tissue Tek, QLD, Australia). 10 μm-thick sections of frozen car were sectioned normal to the skin surface and approximately parallel to projection holes rows using a Leica Ultracut UCT cryo-microtome (Leica Microsystems, Wetzlar, Germany) at the HistoTechnology facility of the QIMR Berghofer Medical Research Institute. Sections were imaged under a Zeiss LSM510 confocal microscope (Carl Zeiss Inc., Germany), using excitation and collection wavelengths of 488 nm and 500-550 nm nm, respectively. The fluorescent tracks left by fluorescent microsphere-coated projections were measured using imageJ (NIH, USA, http://imagej.nih.gov/ij/) for a minimum of 3 slides (distributed uniformly across the treated area) per ear sample, resulting in over 100measurements per application condition. Because penetration depth varied across the array, the measurements taken for each slides were divided in an edge group, including up to 10 tracks from each side, and a center group, including all other tracks. For each slide the mean and standard deviation of the depth measurements was calculated for the edge group and center group independently. A weighted average was performed on the center group means and standard deviation for each slide within a sample, with weights equal to the number of track measured per slide. This allowed the measure to rely more on slides with a larger amount of tracks. The standard deviation was also calculated across the slides within a sample. An identical procedure was followed for the edge group. For each one of the n=4 car samples, the mean and standard deviation between the center and edge group means gave the sample mean and error. The overall mean (across the repeats of each penetration condition) penetration depth (FIGS. 4B, 5A-5E and 6A-B) was further calculated as weighted average across sample means with weights equal to the number of tracks measured in each car, to allow the result to rely more on samples where more tracks were measured. The standard deviation across the samples means was taken as measure of overall standard error (se) of the mean depth and plotted as error-bars (FIGS. 4B, 5A-5E and 6A-B). To quantify the penetration depth variation due to skin (and application) variability across subjects (mice), the standard deviation (of the population) was estimated by multiplying the se of the mean depth by the square root of the number of terms n^{t} in each average step performed, according to the Bienaymé's formula se=sd/(nₜ)^{0.5} (see any inferential statistics textbook). Note that this is a rough approximation because statistical independence of the values in the sample cannot be strictly assumed. This factor is ±2^{0.5}40^{0.5} (where ‘2’ derives from the step where center and edge means were averaged, and ‘40’ is (conservatively) the largest number of tracks measured in an edge or center group). To quantify the penetration depth variation due to microprojection position across the array, the depths of the 10 most peripheral tracks were averaged across slides, and then again across samples. The maximum of such 10 mean depths was taken to be the upper end of the bar in FIG. 4D. Similarly, the depths of 10 center tracks were averaged across slides, and then across samples. The minimum of such 10 mean depths was taken to be the lower end of the bar in FIG. 4D. Separately, cryogenic SEM of penetrated skin was performed in accordance with Coffey et al. (J. W. Coffey, S. R. Corrie, M. A. Kendall, Biomaterials 2013, 34, 9572).

### Example 3

### Indenter/Microprojection Model

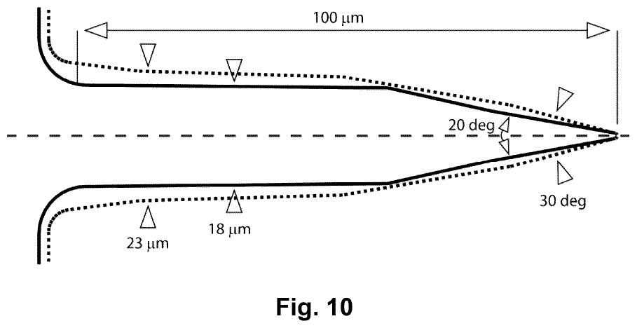

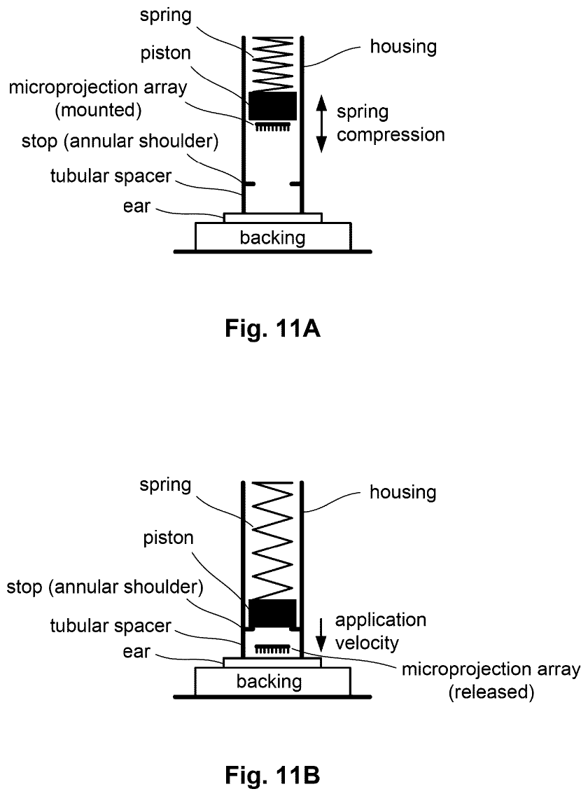

The microprojection geometry was drawn according to the SEM measurements (FIG. 10). The coated profile was considered for the penetration-depth study to accurately reproduce the characteristics of the arrays used for the experimental validation. The microprojections (or indenters) were assumed to be undeformable because silicon (E_{Si}>100 GPa) is over 100-fold stiffer than the skin (M. A. Hopcroft, W. D. Nix, T. W. Kenny, J Microelectromech S 2010, 19, 229). Euler buckling theory (R. C. Hibbeler, in Statics and Mechanics of Materials, Prentice Hall, Singapore 2004) was used to estimate the critical axial load of microprojections ˜40 mN, which is above the maximum force acting on axially on the tip for the application conditions used in this work. Post-application examination of the arrays showed negligible or no microprojection failure.

The motion of the rigid analytical surface that modeled the projection was characterized by an initial velocity (i.e. the velocity generated by the applicator) and a bound mass (determined by the piston mass). The movement was restricted to translation along the vertical axis x=0, y=0, i.e. orthogonal indentation respect to the skin surface. Normal contact interactions were implemented in the FEA using the kinematic contact method because the penalty method was occasionally observed to allow cross-over of the master (microprojection) and slave (skin) surfaces. This happened although the skin elements in contact with the indenter/microprojection were always much smaller than the tip radius (<<0.5 μm). In contrast, the simpler penalty method was used to model tangential friction contact. A friction coefficient of 0.4 was chosen according to the experimental measurement of Bhushan and colleagues (B. Bhushan, J Colloid Interf Sci 2012, 367, 1; B. Bhushan, S. Chen, S. R. Ge, Beilstein J Nanotech 2012, 3, 731).

### Example 4

### FE Parameterization of Skin Fracture

Ultimate and yield strength, and plastic strain at damage were derived from previous works (R. C. Haut, Journal of Biomechanical Engineering-Transactions of the Asme 1989, 111, 136). The properties measured for the SC in high humidity conditions (˜90% RH) where used to parameterize the VE, because the corneocytes are essentially flattened and dried epidermal cells. The properties measured for whole skin were used to parameterize the dermis because this layer dominates the skin overall composition and mechanical properties (R. Reihsner, B. Balogh, E. J. Menzel, Med Eng Phys 1995, 17, 304). For simulations including fracture, the vertical mesh pitch (i.e. element length) was increased in the SC and VE and decreased in the deep dermis to allow larger element deformation and better accuracy in the simulation of dermal penetration.

### Example 5

### Experimental Characterization of Impact Velocity and PDMS Backing Behavior

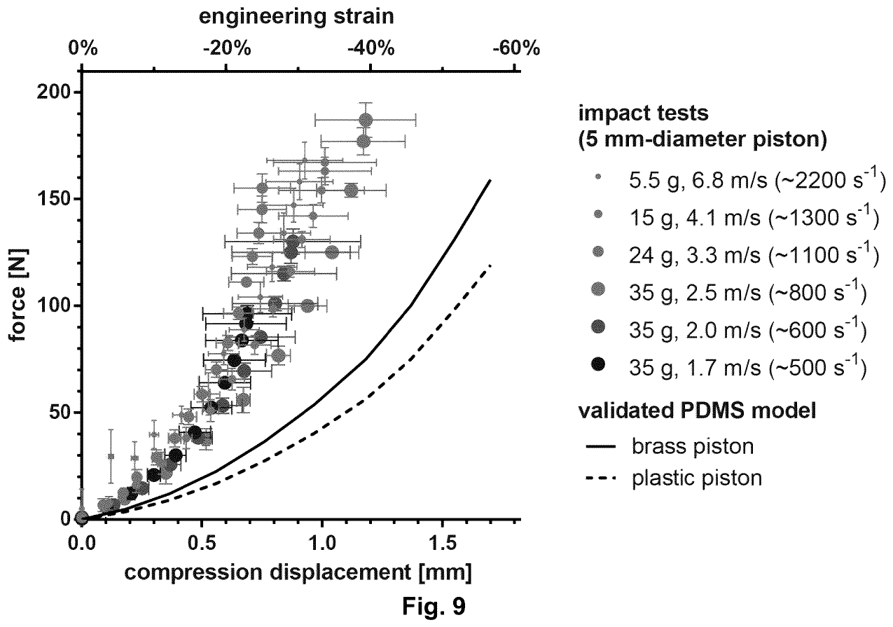

To characterize the impact response of the backing alone, the applicator was fired (n=5) without array on the PDMS+carbon tab (no ear) using different masses and spring compressions (resulting in 1-7 m s⁻¹). The movement of the piston was filmed using a Photron SA4 high-speed camera (HSC) at 20,000 frames s⁻¹ (Photron Inc., San Diego, CA, USA). We tracked the motion of the piston with the HSC software to obtain piston displacement, velocity and acceleration over time before and after contact with PDMS. The dynamic compression displacement of the backing was then the combined with the transient impact force measured (n=5) with a quartz force sensor (model 208C02, PCB piezoelectronic, Depew, NY, USA) placed under the PDMS slab and recorded using a labview program (National Instrument Corp., Austin, TX, USA). The resulting force-displacement characteristic (FIG. 9) was non-linear with a small-strain stiffness ˜20 N mm⁻¹. This was in agreement with dynamic mechanical analysis (DMA) tests (not shown) using an Instron Testing System 5543 (Instron, Norwood, MA, USA) equipped with a 5×5 mm² probe driven at 50 Hz with peak-to-peak amplitude of ˜0.8 mm (i.e. peak displacement velocity ˜0.1 m s⁻¹). The loss tangent was tan δ=0.23 ±0.06 and in the typical range for elastomers and viscoelastic rubbers. Separately, the impact energy U (FIG. 3H) was calculated from the momentum p=(2 Um)^{0.5}, which was obtained integrating the load-cell force-time curves (FIG. 8) up to the peak.

### Example 6

### Backing Lumped-Parameter Model

The backing was modeled as a viscoelastic material using the lumped-parameter Kelvin-Voigt-like element consisting of a mass connected to ground through a spring-damper parallel, and implemented in Abaqus using a connector element. The non-linear stiffness k measured with the impact tests (FIG. 9) was implemented in tabular form. The effective mass m* accounts for the inertia of the mass distributed across the thickness of PDMS itself, hence was approximated to ⅓ of the mass of the PDMS volume covered by the piston according to E. Linder-Ganz, A. Gefen, Mechanical compression-induced pressure sores in rat hindlimb: muscle stiffness, histology, and computational models, Vol. 96, 2004. The damping coefficient is c=tan δ(k m*)^{0.5}, where k was approximated to the small-strain value. This model (backing only) was employed to simulate the backing impact test and the parameterization iteratively refined until the numerical force response matched the results of the backing impact tests. All lumped parameters were scaled according to the area simulated when used in conjunction with the skin FE model, i.e. m*, k and c relative to the piston impact tests where divided by the piston cross-sectional area and multiplied by the square of the microprojection spacing.

### Example 7

### The Out-of-Plane Hyperelastic Properties of Skin Layers for Varying Strain Rates

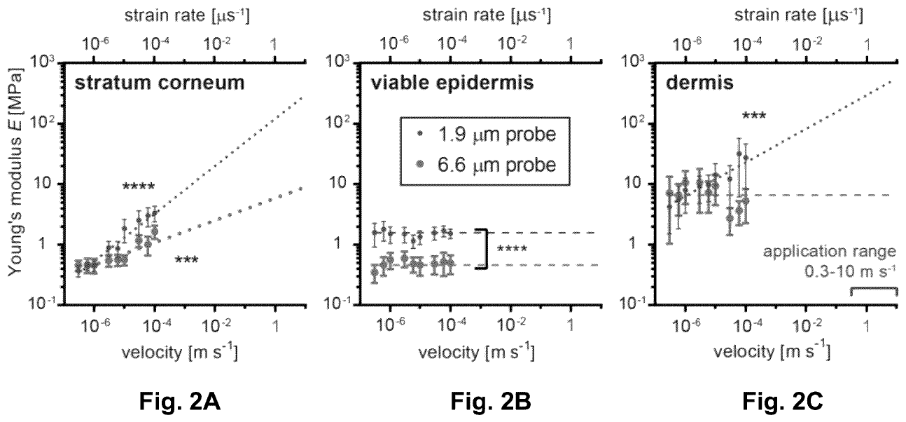

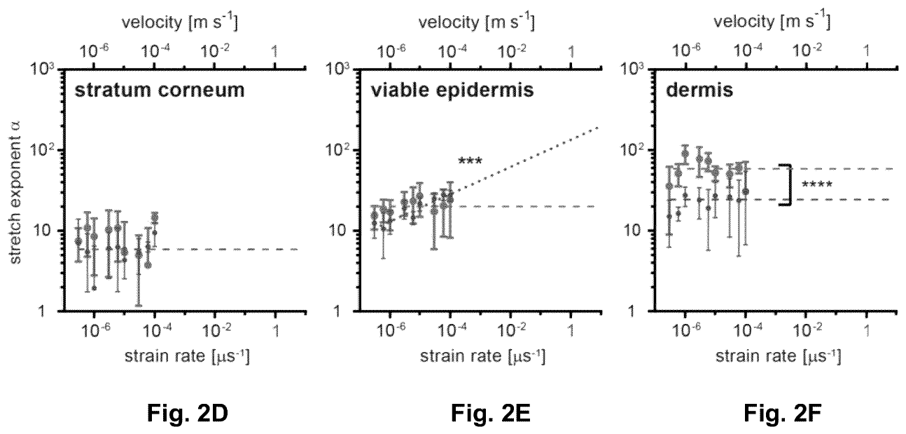

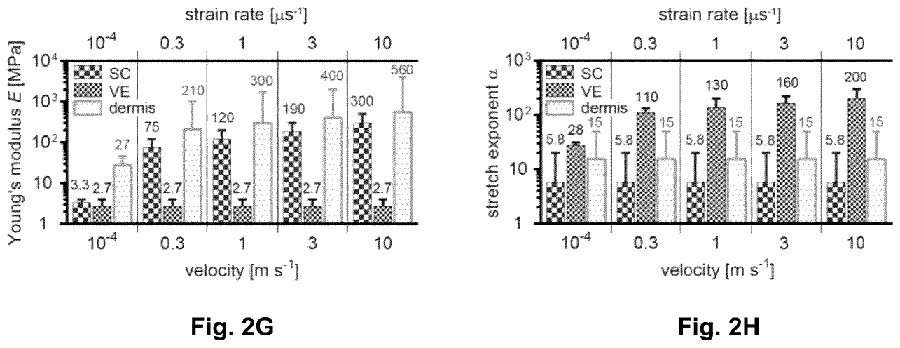

The strain-rate dependence of skin elasticity by indenting individual strata of freshly-excised mouse ear (SC, VE and dermis) with spherical tips (1.9 μm and 6.6 μm in diameter) at different velocities was investigated. This experimental procedure and the extrapolation hyperelastic 1st-order Ogden parameters was performed as described by M. L. Crichton, B. C. Donose, X. F. Chen, A. P. Raphael, H. Huang, M. A. F. Kendall, Biomaterials 2011, 32, 4670 (FIG. 2A-2F). In FIGS. 2A-2F, the purple data were collected with a 1.9 μm probe and the green data were collected with a 6.6 μm probe. The approximate logarithmic strain-rate generated is indicated by the top abscissa. A dotted line indicates that a statistically significant Spearman correlation was found between the hyperelastic parameter and the velocity/strain rate, and represents a linear regression in Log-Log scale. A horizontal dashed line indicates that the correlation was not significant (p>0.05). A square bracket indicates a statistically significant variation of the hyperelastic parameter with probe size; **** p<0.0001, *** p<0.001. Young's modulus E of the SC (both probe sizes; FIG. 2A) and dermis (small probe only; FIG. 2C), and the stretch exponent α of the VE (small probe only; FIG. 2E) significantly correlated (Spearman r≥0.95, p<0.001) with the indentation velocity. This further implicates correlation with the peak strain rate at contact because of its defining linear relationship with the probe impact velocity. Power relationships, i.e. the dotted straight lines in Log-Log scale, fitted these datasets better (adjusted R²>0.83 except for SC 6.6 μm-probe E that scored 0.62) than logarithmic, linear and exponential curves. This rate dependency is in general agreement with the elastic properties previously extrapolated from in-plane uniaxial stretch tests on pig skin up to ˜10⁻² μs^{-1[45]} and rat skin up to ˜10⁻⁴ μs⁻¹. For the parameters that correlated with velocity non-linear regressions were used to predict the layer hyperelastic properties at larger strain rates (0.3-10 μs⁻¹), i.e. relevant for the application microprojection arrays (0.3-10 m s⁻¹). For example, FIG. 2G shows that the Young's modulus of the SC and dermis increase with strain rate and is expected to exceed 100 MPa above 1 μs⁻¹, whereas it remains approximately constant and below 5 MPa for the VE. FIG. 2H indicates that the stretch exponent (α) of the VE may increase over 100 at strain rate >1 μs⁻¹. No previous report of such effect was found for the skin. In FIGS. 2G and 2H, both the column height and the numbers indicate the means; the error bars represent the se for the experimental measurement at 10⁻⁴ m s⁻¹, whereas show the 90% prediction band for the values extrapolated at 0.3-10 m s⁻¹.

Separately, the smaller tip resulted in a statistically significant (Wilcoxon p<0.0001) larger E for the VE (FIG. 2B) and lower α for the dermis (FIG. 2F), compared to the larger tip. Recent measurements of whole mouse car skin showed an inverse Log-Log linear trend (Eₛₖᵢₙ=29×(2r)⁻¹; Eₛₖᵢₙ in MPa, r in μm) between the Young's modulus and the probe radius r across μm to mm scales. The analogous curve (not shown) intercepting our two scale-dependent values of VE Young's modulus (averaged over the velocities) was E_{VE}=2.7×(2r)^{-0.9}. SC stretch exponent did not show significant scale or rate dependence (FIG. 2D), thus the overall mean across the velocities for the small probe was reported in FIG. 2H.

### Example 8

Skin Failure and Fracture Mechanics during Penetration: Model and Properties

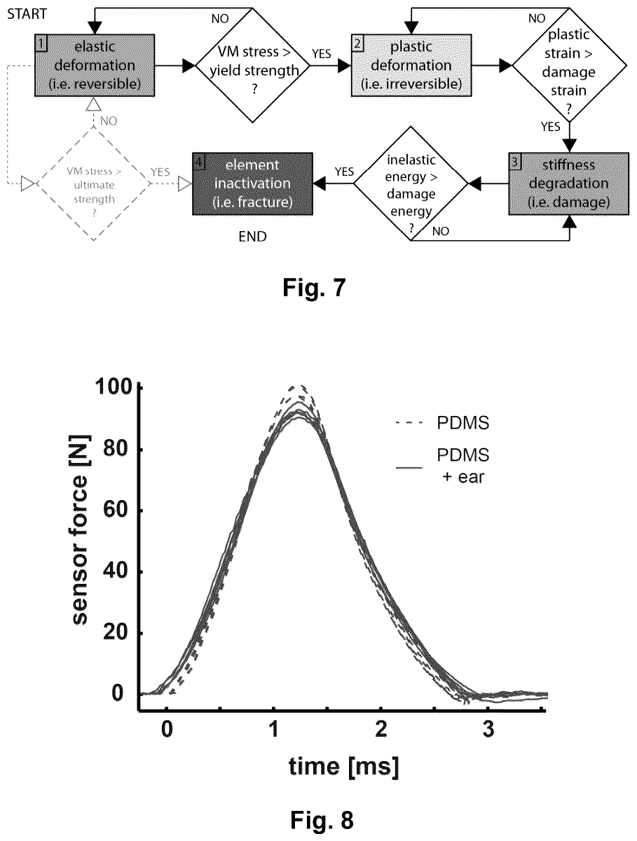

Characterization of skin penetration following penetrator impact was accomplished by numerically modeling microprojection application to skin and comparing against experimental observations. FIG. 7 illustrates the descriptive framework used to capture skin failure and fracture mechanics. In brief, 1) a skin element deforms reversibly according to the hyperelastic properties; 2) when the von Mises (VM) stress exceeds the yield strength, it starts deforming irreversibly (plastically) according to a linear curve (dotted) that intercepts the stress-strain coordinate defining the onset of damage (breaking strength and strain at damage); 3) when the plastic strain exceeds a damage threshold, the skin element progressively loses stiffness (material damage) linearly with the plastic energy dissipated (dashed line); 4) the element is completely inactivated when this plastic energy reaches a characteristic fracture energy.

The initial values for the failure properties were determined starting from previous skin mechanical tests and then refined to validate the fracture model against the penetration experiments. The puncture and tearing energy of whole skin and isolated SC has been reported to exceed 600 pJ μm⁻². Initially, simulation of a 2 m s⁻¹ microprojection impact using the threshold strengths and strains and fracture energy of 600 pJ μm⁻² for all skin layers resulted in failure initiation and plastic deformation of the elements. However, no element inactivation occurred above 6 μm displacement of the tip into the skin, with a maximum stiffness degradation <10%. This indicated that the fracture energy had been overestimated, possibly because previous measurements could not isolate fracture dissipation from other energetic contributions (e.g. elastic strain or yielding). Hence, we varied the layer fracture energies in the range 0-200 pJ μm⁻² (0, 0.2, 1, 6, 35, 100 and 200 pJ μm⁻² were used) until the simulations matched the fracture behavior observed experimentally. For example, the SC optimal energy was approximately 35 pJ μm⁻² suggesting that its rupture occurs through a combination of delamination (energetically ‘cheaper’ 1-10 pJ μm⁻²) and tear (energetically more ‘costly’ ˜10³ pJ μm⁻²). Using the layers optimal energies, the total irreversible strain energy (i.e. plastic and damage dissipations) when the projection has penetrated to the bottom boundary of the dermis (i.e. 4.45 μs after the contact) was about 100 nJ. The simulations showed that this value was most sensitive to the dermis fracture energy, probably due to its larger thickness. The dissipation error bounds were taken to be 50 nJ and 170 nJ, which resulted when the dermis was parameterized with 1 pJ μm⁻² and 35 pJ μm⁻², respectively. Such error range is reasonably tight compared to the total energy of the system (the application energy per projection is 21 μJ) and is satisfactory for the purpose of this work considering the limited literature about rupture energy measurements, especially for penetration-like fracture modes.

SC flaps partially overlap with the VE. This non-physical behavior occurs because, for simplicity, no ‘self’-contact interaction properties were defined for the skin elements. However, the overlap involves skin portions that have already failed and have little or no load-bearing capacity; therefore, the errors in strain energy and stress were assumed to be negligible. Interestingly, stiffness degradation and fracture (element inactivation) originated ˜1 μm off the microprojection axis, i.e. where the dilatational strain peaked, rather than immediately below the tip where the VM stress and compressive strain peaked. This also indicates that this fracture approach captures, at least in part, the different rupture behaviors in tension and compression, in contrast with fracture models solely based on a VM stress threshold. Note that the cartilage was not assigned failure mechanisms because this work focuses on skin targeting and cartilage penetration is avoided. Rather, to avoid bias of the numerical results due to artificial cartilage resistance to penetration, the projection was allowed to penetrate the cartilage with at zero energy cost by deactivating contact interactions of its FE nodes with the microprojections. Having established the optimal skin fracture parameters, this failure implementation is used in the next section to simulate the penetration by arrays of microprojections.

### Example 9

### Energy Contributions to Skin Penetration: Elastic Deformation, Fracture and the Role of Subcutaneous Backing Layers

FIG. 3A represents snapshot along the penetration trajectory of a ˜3000-microprojection array impacting the skin at 2 m s⁻¹ with a bound mass (applicator piston) of ˜35 g. According to this simulation, when a microprojection has penetrated to the dermis bottom boundary its velocity has decreased negligibly (<2%) and penetration would continue across the cartilage. FIG. 3F shows that less than 3% of the initial application energy is transferred to the skin, while the majority remains array kinetic energy. In contrast, experiments showed that similar application velocities (˜2 m s⁻¹) result in mid-to deep-dermal penetration. This means that the current model does not account for several mechanisms that absorb a major fraction (>90%) of the application energy. One possible reason could be attributed to the linked assumption that microprojections are largely spaced and do not influence each other. Hence, the penetration of arrays with finite microprojection densities/spacings (FIG. 3B-D) was simulated using the 3D symmetric FE geometry schematized in FIG. 3E. Interestingly, densities around 10 kproj cm⁻² (i.e. 10,000 proj cm⁻²) appeared to decrease the friction dissipation in favor of an increased energy contribution to failure and fracture (FIG. 3G). The elastic strain energy was approximately constant with the projection density; however, VM stress above 1 MPa concentrates at the penetration site in the ventral (top) skin layers when the projections are largely spaced, while it progressively spreads to the cartilage, dorsal (bottom) dermis, VE and SC as the density approaches 20 kproj cm⁻². Most importantly, the total energy transferred to the skin when the projection has penetrated to the bottom of the dermis is essentially independent of the microprojection density (at least up to 20 kproj cm⁻²). Rather, the remaining kinetic energy may be transferred to the backing layer, i.e. a 3 mm-thick PDMS slab placed under the car during the microprojection array application. This is employed to cushion the impact and avoid ear tissue damage while allowing applications at high velocities (˜m s⁻¹). The force transmitted across the backing was measured by placing a piezoelectric load cell below the PDMS slab (i.e. on the bench support; FIG. 8). FIG. 3H shows that this energy is approximately 5% lower than the energy transmitted when a flat (projection-less) patch is applied on the backing alone (without mouse car). This means that only a small amount of energy (˜5%) is transferred to the ear, which explains the excess of energy in the simulation (˜95% to the backing). Hence, accurate modeling of skin penetration requires accounting for possible compliant backing layers like our PDMS or subcutaneous fat and muscle found in vivo (less stiff than skin).

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

1. An apparatus for delivering an active ingredient into the skin of an animal at a defined depth, the apparatus including:
a) a microprojection array including a plurality of microprojections having a density of 2000 projections per cm² to 5000 projections per cm²; and,
b) an applicator for driving the microprojection array towards the skin in use so that the microprojection array impacts on the skin with a mass-to-velocity ratio of between 0.005 g/m/s and 0.5 g/m/s wherein the mass is the mass of the microprojection array.

2. The apparatus of claim 1, wherein the microprojection array has a mass between 0.001 g and 0.05 g.

3. The apparatus of claim 1, wherein the microprojection array is configured to impacts the skin at velocities between 10 m/s and 50 m/s.

4. The apparatus of claim 1, wherein the microprojection array has an area between 10 mm² and 40 mm².

5. The apparatus of claim 1, wherein the microprojections are solid.

6. The apparatus of claim 1, wherein the microprojections are tapered and hexagonal.

7. The apparatus of claim 6, wherein the microprojections have a length of between 200 μm and 300 μm.

8. The apparatus of claim 7, wherein the microprojections include:
a) a base having a width of about 5 μm to about 50 μm; and,
b) a tip having a width of 0.5 μm to 2 μm.

9. The apparatus of claim 1, wherein the applicator includes a driver configured to drives the microprojection array towards the skin and wherein the microprojection array is releasably mounted to the driver so that the microprojection array is configured to be released from the driver prior to the microprojections contacting the skin.

10. The apparatus of claim 9, wherein the driver abuts against a stop to configured to thereby release the microprojection array from the driver.

11. The apparatus of claim 9, wherein the stop includes an annular shoulder.

12. The apparatus of claim 9, wherein the applicator includes:
a) a housing containing the driver; and,
b) a substantially tubular spacer that in use is positioned with an open end in contact with a surface of the skin to thereby space the housing from the skin, the stop being provided proximate the open end of the spacer.

13. The apparatus of claim 9, wherein the driver is configured to be urged from a retracted to an extended position using a biasing mechanism, and wherein the biasing mechanism and engagement between the driver and a housing define a driver velocity in use.

14. The apparatus of claim 13, wherein the driver is a piston.

15. The apparatus of claim 13, wherein the biasing mechanism is a pneumatic actuator.

16. The apparatus of claim 14, wherein the engagement is frictional engagement between a piston and piston chamber within the housing.

17. The apparatus of claim 1, wherein the microprojection array is configured to impacts on the skin with a mass-to-velocity ratio sufficiently high to dissipate inertia so as to avoid mechanical stress on body parts underlying the skin and cause a controlled amount of mechanical stress for immune-enhancing inflammation.

18. The apparatus of claim 1, wherein at least tips of the microprojections are coated.

19. The apparatus of claim 1 wherein the active ingredient is one or more vaccine antigens.

20. The apparatus of claim 8, wherein the base has two substantially parallel sides with a draught angle of 1 to 20 degrees up to a transition point at which point the draught angle increases from 20 to 70 degrees.
